(19) United States
(12) Patent Application Publication
(10) Pub. No.: US 2012/0244213 A1
(43) Pub. Date: Sep. 27, 2012

(54) METHODS FOR THE TREATMENT OF SPEECH IMPEDIMENTS

(76) Inventor: Liora Emanuel, Tel Aviv (IL)

(21) Appl. No.: 13/511,788

(22) PCT Filed: Dec. 14, 2010
(86) PCT No.: PCT/IL2010/001061

§ 371 Date: May 24, 2012

Related U.S. Application Data
(60) Provisional application No. 61/287,220, filed on Dec. 17, 2009.

(51) Int. Cl. A61K 38/11; A61P 25/00; A61P 21/00; A61K 9/127; A61P 43/00

(52) U.S. Cl. 424/450; 514/11.6

(57) ABSTRACT

The present invention relates to methods of treating for treating stuttering or other speech impediments that are not related to psychiatric disorders or autism using oxytocin or its analogs.

### FIELD OF THE INVENTION

The present invention relates to therapeutic methods for treating stuttering or other speech impediments that are not related to psychiatric disorders or autism using oxytocin or its analogs.

### BACKGROUND OF THE INVENTION

Stuttering is a disturbance in the normal fluency and time patterning of speech that is inappropriate for the individual's age, and is characterized by frequent occurrences of sound repetitions, prolongations and/or blockings among other speech impediments, which may be accompanied with involuntary movement of facial muscles or other body muscles induced by circumlocusions and physical tension.

Conditions that trigger stuttering vary greatly but mostly involve social anxiety, including the general form of social anxiety and performance anxiety. Additional causes may be physical, psychological, environmental, cognitive or a combination thereof.

The physical condition may be a predisposition of a difficulty to synchronize speech organs while using them for speech which may result with negative habits of responding to speech situations. Negative habits are usually developed early in life and thus are difficult to overcome.

Commonly known treatments to overcome stuttering are speech therapy techniques for developing speech fluency addressing behavioral aspects including changing attitudes interventions like Cognitive Behavior Therapy (CBT), Rational Emotive Therapy (RET) and Solution Focused Therapy (SFT). The problem underlying these techniques is the difficulty in changing speech habits, especially for adults. Moreover, stuttering patients often find it hard to implement fluency techniques or change of attitude in real life because of social anxiety.

A medicinal approach for treating a tic disorder associated with stuttering is disclosed and claimed in U.S. Pat. No. 7,098,230. The '230 patent teaches administering a specific dopamine uptake inhibitor for treating disorders, inter alia, a tic disorder, which are alleviated by inhibiting dopamine uptake.

Reissued US Patent No. RE 39,030 discloses the use of fluoxetine, an anti-depressant commercially known as Prozac®, for treating a variety of psychiatric disorders, including stuttering.

Oxytocin was one of the first peptide hormones to be isolated and sequenced. It is an extremely short-lived, fast acting hormone, made by the hypothalamus, stored in the posterior pituitary, and released into the blood upon need. It stimulates certain smooth muscle cells, responsible for special vascular constriction, including vascular constriction that lessens placental separation bleeding and facilitates the sensitivity of some tissues to other hormones and nerves. Particular physiological effect of oxytocin relate to uterine muscle contractions where of the most common uses of oxytocin are for stimulation of uterine contraction to induce labor, for the control of postpartum hemorrhage following delivery of the placenta and for stimulation of lactation for breast-feeding. Oxytocin is currently prepared synthetically and sold under various trade names including Pitocin™. (Parke-Davis) and Syntocinon™ (Novartis Pharmaceuticals).

In recent years oxytocin was found to be useful in enhancing prosocial behavior (Petrovic et al., Emotion, December 2008, vol. 8(6): 781-791) and in reducing stress and fear (Kosfeld et al., Nature, 435:673-676, 2005; Kirsch et al., The Journal of Neuroscience, 25(49):11489-11493, 2005). Oxytocin was also suggested for the treatment of neurological and psychiatric disorders, particularly, autism, as disclosed for example in US 2006/0105939, US 2007/0032410, WO2008/042452, WO 2008/150305 and JP 2002322085.

Nowhere in the background art is it taught or suggested that oxytocin would be effective in treating stuttering and any other speech impediment, specifically, non-psychiatric speech impediments. Moreover, there is an unmet need for an effective medicinal treatment of stuttering.

### SUMMARY OF THE INVENTION

The present invention is directed to methods for treating stuttering and any other speech impediments that are not related to psychiatric disorders or autism, by using oxytocin or its analogs.

The methods of the invention are advantageous over speech and behavioral therapies commonly used for treating speech impediments. Successful therapeutic outcome of speech and behavioral therapies is subject to patient's ability to implement the therapeutic techniques in daily life. However, changing old habits of speech or behavioral pattern is a complicated task at all ages. Moreover, patients find it hard to implement fluency techniques or change of attitude techniques in real life situation, where they are also under some form of social anxiety. The methods of the invention overcome these problems as they do not require a trained response from the subject and are independent of the surrounding. Rather, the methods of the invention merely require the compliance expected of any patient in need of drug therapy. The method of the invention is based on a medicinal approach, using a well known peptide hormone naturally synthesized in the body and commonly used in the clinic for a vast range of treatments other than for treating speech impediments, such as stuttering, that are unrelated to psychiatric causes.

According to one aspect, the present invention provides a method of treating stuttering in a patient in need thereof, comprising administering to the patient a composition comprising a therapeutically effective amount of oxytocin or an analog thereof.

According to another aspect, the present invention provides a method of treating speech impediments unrelated to psychiatric disorders or autism in a patient in need thereof, comprising administering to the patient a composition comprising a therapeutically effective amount of oxytocin or an analog thereof.

It is to be understood that the term ‘stuttering’ is interchangeable with any of the terms ‘stammering’, ‘speech dysfluency’ and ‘speech impediment’, specifically speech impediment unrelated to psychiatric disorders or autism.

According to one embodiment, the oxytocin is an oxytocin analog selected from the group consisting of: 4-threonine-1 -hydroxy-deaminooxytocin, 9-deamidooxytocin, 7-D-proline-oxytocin and its deamino analog, (2,4-diisoleucine)-oxytocin, deamino oxytocin analog, 1-deamino-1-monocarba-E12-[Tyr (OMe)]-OT(dCOMOT), carbetocin, Ile-conopressin, atosiban, [Thr4-Gly7]-oxytocin, oxypressin, deamino-6-carba-oxytoxin (dC60), d[Lys(8)(5/6C-Flu)]VT, d[Thr(4), Lys(8)(5/6C-Flu)]VT, [HO(1)][Lys(8)(5/6C-Flu)]VT, [HO(1)][Thr(4), Lys(8)(5/6C-Flu)]VT, d[Om(8)(5/6C-Flu)]VT, d[Thr(4), Om(8)(5/6C-Flu)]VT, [HO(1)][Om(8)(5/6C-Flu)]VT, [HO(1)][Thr(4), Om(8)(5/6C-Flu)]VT and oxytocin fragments.

According to another embodiment, the method comprises administering the composition in a route selected from transmucosal, sublingual, by inhalation and transdermal.

According to yet another embodiment, the composition of the invention is administered transdermally by means selected from the group consisting of: iontophoresis, hydrogels, patches, gels, and liposomes. According to yet another embodiment, the composition of the invention is administered by transmucosal administration selected from the group consisting of: buccal, nasal and sublingual. According to yet another embodiment, the composition of the invention is administered by subcutaneous injection.

According to yet another embodiment, the composition of the invention is administered by nasal or oral inhalation.

In yet another embodiment the composition of the invention is delivered in a modified release manner.

The term “modified release” as used herein is interchangeable with any of the terms “time-dependent release”, “extended-release”, “sustained release”, “controlled-release” or “continuous-release” and refers to a mechanism by which the active ingredient is released over time as opposed to an immediate release form.

According to yet another embodiment, the method of the invention is combined with one or more therapies selected from speech therapy and behavioral therapy.

According to yet another aspect, the present invention provides use of a composition comprising a therapeutically effective amount of oxytocin, an oxytocin analog or a combination thereof for the preparation of a medicament for stuttering.

According to one embodiment, the medicament is for inhalation. According to another embodiment, the composition is a modified release composition.

According to yet another aspect, the present invention provides use of a composition comprising a therapeutically effective amount of oxytocin, an oxytocin analog or a combination thereof for the preparation of a medicament for speech impediments unrelated to psychiatric disorders or autism.

According to yet another aspect, the present invention provides a pharmaceutical composition comprising a therapeutically effective amount of oxytocin or an analog thereof for the treatment of stuttering.

According to yet another aspect, the present invention provides a pharmaceutical composition comprising a therapeutically effective amount of oxytocin or an analog thereof for the treatment of speech impediments unrelated to psychiatric disorders or autism.

Further embodiments and the full scope of applicability of the present invention will become apparent from the detailed description given hereinafter. However, it should be understood that the detailed description and specific examples, while indicating certain embodiments of the invention, are given by way of illustration only, since various changes and modifications within the spirit and scope of the invention will become apparent to those skilled in the art from this detailed description.

### DETAILED DESCRIPTION OF THE INVENTION

The present invention relates to methods and uses for treating stuttering or other speech impediments that are not related to psychiatric disorders or autism using oxytocin or its analogs.

According to the Diagnostic and Statistical Manual of Mental Disorders (DSM IV), stuttering is characterized by frequent occurrences of one or more of the following: (i) sound and syllable repetitions; (ii) sound prolongations; (iii) interjections; (iv) broken words (e.g., pauses within a word); (v) audible or silent blocking (filled or unfilled pauses in speech); (vi) circumlocutions (word substitutions to avoid problematic words); (vii) words produced with an excess of physical tension; and (viii) Monosyllabic whole-word repetitions. A ninth characteristic recently suggested by Maguire and Bernstein-Ratner, (Aug. 6, 2009 IFA Convention) is avoidance and/or anxiety around feared speaking situations or words.

The term ‘stuttering’ as used herein is synonymous with the terms ‘stammering’ ‘speech disfluency’ and ‘speech impediment’ so long as the speech impediment is not a symptom of a psychiatric disorder or autism. Accordingly, the method of the present invention is directed to the treatment of stuttering and any speech impediment associated with non-psychiatric disorders or autism, including, but not limited to, dyspraxia, dysarthria, dysphasia, selective mutism and the like.

There is a strong need for an effective treatment of stuttering as this disorder, from which 1% of the world's adult population and 5% of the world's child population suffer, interferes with the most important aspects of life, namely, occupational (including academic) achievement and/or with social communication.

So far the known treatments for stuttering are speech techniques, attitude-change techniques and medicinal therapies which are directed to psychiatric and neurologic disorders involving, inter alia, stuttering. To date, there is no medicinal therapeutic treatment that is directed to stuttering or any speech impediment that is not associated with a psychiatric condition.

The present invention provides for the first time a novel application of oxytocin, namely, use of this hormone for treating stuttering that is not involved in a psychiatric disorder or autism.

Without being bound by any method or mechanism, it is believed that most speech impediments that are not related to psychiatric disorders or autism, such as stuttering, relate to social anxiety, whether general or performance related anxiety. It has been shown that social anxiety is associated with activation or inhibition of certain basic brain circuits, in particular, marked activation of limbic regions such as the amygdala and a failure of frontal regions to give a logical or appropriate assessment of what the realistic harm is. Thus, in a sense, social anxiety is an outcome of an imbalance between the amygdala and the frontal regions. The association of stuttering with social anxiety is one way to explain the failure of speech and behavior therapies in many cases of stuttering: the ability of a stuttering patient to practice these techniques decreases significantly under social anxiety. A particular case of stuttering which clearly relates social anxiety to stuttering is the Covert Stuttering disorder. Patients suffering from Covert Stuttering have the ability to “hide” the majority of stuttered moments. However, these patients report major anxiety feelings in speech situations.

Oxytocin is a peptide discovered in 1909 having two cysteine residues that form a disulfide bridge between positions 1 and 6 (Baulieu and Kelly, Hormones: From Molecules to Diseases, Hermann Publishers; 1990, 283).

It is the neurohormone which is responsible for initiating childbirth and the letdown reflex in lactating women and is released during sexual orgasm. Oxytocin has been thought of as an affiliation hormone because research on nonhuman mammals has demonstrated that it plays a key role in the initiation of maternal behavior and the formation of adult pair bonds. Oxytocin is also known to play a central role in bonding mothers to their offspring and in social behavior generally. It was also demonstrated, by way of brain-imaging studies in humans, that oxytocin damps down activity in the right side of the amygdala, a part of the brain that processes emotional responses and in particular most responsive to anxiety.

The main tissues affected by oxytocin are: uterus, including endometrium and myometrium, vaginal, breast (both sexes), erectile and seminal vesicles. Oxytocin has special effects on uterine muscle contractions in both birth and orgasm, the vascular constriction that lessens placental separation bleeding, and the let-down reflex induced in nursing mothers when babies cry.

Aside from the well known therapeutic uses of oxytocin (e.g. inducing delivery) it has been recently shown to be useful in treating psychiatric and neurological disorders (e.g. US 2007/0032410), to enhance prosocial behavior (Petrovic et al. ibid), and to induce reduction in repetitive behaviors of patients with autism spectrum disorders (Hollander et al. Neuropsychopharmacology, 28:193-198, 2003). All these uses are substantially different from the methods of the invention. Specifically, the method of the invention is drawn to use of oxytocin for treating stuttering and other speech impediment that do not associate with psychiatric and neurologic disorders, including, the aforementioned disorders and autism.

Although autism is a disease that affects millions of people, there is no simple way to define autism. One definition is that autism is a brain disorder resulting in communication, socialization and developmental problems for the individual that is suffering from it.

Another way to define autism is by the characteristics that those with the disease develop. These are basically three types of signs and symptoms, social interaction, verbal and non-verbal communication and symbolization, and restricted areas of interest. Not every individual that has autism carries the same signs and symptoms as anybody else with the disease. For that reason, it is often necessary to recognize at least one or two of the symptoms in order to be able to detect it early.

However, it is to be understood that use of oxytocin for treating autism is known, while the present invention is directed to use of oxytocin for treating non-autistic patients. In particular, the methods of the invention are directed to self-administration of oxytocin and therefore are not suitable for most autistic patients.

The composition of the invention comprises a therapeutically effective amount of oxytocin, an oxytocin analog or a combination thereof, and a pharmaceutically acceptable carrier.

The phrase “pharmacologically acceptable” refers to molecular entities and compositions that do not produce an unacceptably frequent adverse, allergic or other untoward reaction when administered to an animal, or a human, as appropriate.

Additionally, the term “therapeutically effective dose” or “therapeutic dose” refers to the administration of a quantity that renders a therapeutic effect, rather than an adverse, allergic or other non-favorable reactions when administered to an individual, such as an animal, or human.

An example of a therapeutically effective amount of oxytocin, oxytocin analog or combination thereof is 1-100 ug/mL, for a composition which is appropriate for i.v. administration. Alternatively, the active therapeutic agents may be formulated within a mixture to comprise about 0.0001 to 1.0 milligrams, or about 0.001 to 0.1 milligrams, or about 1.0 to 100 milligrams or even about 0.01 to 1.0 grams per dose or so. Multiple doses can also be administered.

In one embodiment, the oxytocin composition is provided in a solution for intravenous administration. An administration regime may include infusing the composition an individual for 3-6 hours, the dose rate of said solution initially may be at about 0.01-1 unit per hour to increase every 15 minutes of the first hour by 0.1 to about 0.5 units per hour, and, optionally, increasing every 15 minutes of the second hour by about 0.2 to about 1 unit per hour, and, optionally, increasing every 15 minutes of the third hour by 1 unit per hour and said dose rate of said solution to remain constant at 7 units per hour the fourth hour. This i.v. method of administration is less favorable in the context of the present invention but would be useful for testing the optimal doses for treating stuttering.

Suitable compositions of the present invention include, but not limited to, hydrogels, patches, gels, and liposomes. All such compositions are well known in the art.

Compositions suitable for transdermal administration include ointments, gels, patches and injections, including powder injections.

In addition, the composition of the invention may be incorporated into modified-release preparations and devices. Modified release preparations include, but are not limited to, time-dependent release preparations, extended-release preparations, controlled-release preparations or continuous-release preparations. Such preparations deliver the active ingredient slowly over time. The advantages of modified-release preparations are that they can often be taken less frequently than immediate-release formulations of the same drug, and that they keep steadier levels of the drug in the bloodstream.

In one embodiment, the present invention provides a pharmaceutical composition comprising a therapeutically effective amount of oxytocin or an analog thereof for the treatment of stuttering. In another embodiment, the present invention provides a pharmaceutical composition comprising a therapeutically effective amount of oxytocin or an analog thereof for the treatment of speech impediments unrelated to psychiatric disorders or autism.

Other formulations which may be suitable for other modes of administration, include suppositories and pessaries. A rectal pessary or suppository may also be used. Suppositories are solid dosage forms of various weights and shapes, usually medicated, for insertion into the rectum or the urethra. After insertion, suppositories soften, melt or dissolve in the cavity fluids. For suppositories, traditional binders and carriers generally include, for example, polyalkylene glycols or triglycerides; such suppositories may be formed from mixtures containing the active ingredient in the range of 0.5% to 10%, preferably 1%-2%.

Methods for administering the oxytocin or oxytocin analog composition of the present invention include, but are not limited to, intranasal administration, sublingual administration, administration by inhalation, buccal administration, transdermal administration, subcutaneous injection and transmucosal administration. All such methods of administration are well known in the art.

Without being bound to any theory or mechanism, it is assumed that oxytocin acts in the central nervous system (CNS) and thus in order to achieve effective treatments with oxytocin, this drug has to cross the blood-brain barrier (BBB). The olfactory mucosa is one of the few areas of the central nervous system which lack a traditional BBB and drug transport directly from the nasal cavity by ways of nasal delivery, into the brain tissue or cerebrospinal fluid has been considered as an efficient tool for systemic administration of drugs acting in the CNS.

As such, a preferred route of administration of the composition of the invention is transmucosal delivery, which includes, but is not limited to, buccal, sublingual, oral and nasal delivery. Aside from the specific benefit related to obtaining an effective treatment with oxytocin, additional advantages of transmucosal delivery include rapid onset of drug delivery, sustained drug delivery levels and rapid decline of drug delivery at the end of the desired treatment time. The transmucosal route also delivers active agent directly into the system and avoids the first-pass through the liver metabolism process which is characteristic of oral delivery. The transmucosal membranes also appear to allow the passage of relatively high molecular weight drugs, including peptides.

For transmucosal administration, any available methods and devices known in the art may be applied. According to a particular embodiment, the autoadhesive oral transmucosal delivery device as disclosed in U.S. Pat. No. 6,488,953 or a gum pad as disclosed in U.S. Pat. No. 6,319,510, may be used. In fact, buccal delivery of oxytocin has been achieved in the past (Syntocinon™, Sandoz; Martindale 29th Edition, p1147). Other technologies available which rely on the adhesion of a solid sustained delivery device to the mucosal membranes to allow sustained delivery of active agent are suitable here. Such systems generally comprise an adhesive layer and a delivery matrix including the active agent, typically in the form of a small tablet or patch. In the Cydot™ system the patch is non-degradable and must be removed after use. In the Theratech™ system the tablet dissolves after a number of hours in situ.

Hydrogels are known sustained release delivery vehicles. A typical hydrogel having well documented pharmacological acceptability is disclosed in GB2047093 and GB2047094. This hydrogel consists of cross-linked polyurethane materials having the ability to swell and absorb several times their own weight of water and is available under the trademarks Propess and Cervidil.

In another preferred embodiment, the composition of the invention is administered by intranasal or intraoral administration, using appropriate solutions, such as nasal solutions or sprays, aerosols or inhalants. Nasal solutions are usually aqueous solutions designed to be administered to the nasal passages in drops or sprays. Nasal solutions are prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations, and appropriate drug stabilizers, if required, may be included in the formulation. Various commercial nasal and oral preparations for inhalation, aerosols and sprays are known and include, for example, antibiotics and antihistamines and are used for asthma prophylaxis.

For intranasal or intraoral administration the composition of the invention is provided in a solution suitable for expelling the pharmaceutical dose in the form of a spray, wherein a therapeutic quantity of the pharmaceutical composition is contained within a reservoir of an apparatus for nasal or intraoral administration. The apparatus may comprise a pump spray device in which the means for expelling a dose comprises a metering pump. Alternatively, the apparatus comprises a pressurized spray device, in which the means for expelling a dose comprises a metering valve and the pharmaceutical composition further comprises a conventional propellant. Suitable propellants include one or mixture of chlorofluorocarbons, such as dichlorodifluoromethane, and the more recent and preferred hydrofluorocarbons, such as 1,1,1,2-tetrafluoroethane (HFC-134a) and 1,1,1,2,3,3,3 -heptafluoropropane (HFC-227). Suitable pressurized spray devices are well known in the art and include those disclosed in, inter alia, WO 92/11190, U.S. Pat. No. 4,819,834, U.S. Pat. No. 4,407,481 and WO 97/09034, when adapted for producing a nasal spray, rather than an aerosol for inhalation, or a sublingual spray. Suitable nasal pump spray devices include the VP50, VP70 and VP100 models available from Valois S.A. in Marly Le Roi, France and the 50, 70 and 100 ul nasal pump sprays available from Pfeiffer GmbH in Radolfzell, Germany, although other models and sizes can be employed. In the aforementioned embodiments, a pharmaceutical dose or dose unit in accordance with the invention can be present within the metering chamber of the metering pump or valve.

Yet, another preferred route of administration is transdermal administration. One example of transdermal delivery system which is suitable for administration of the composition of the invention is an iontotherapeutic device such as provided by U.S. Pat. No. 5,961,482. Iontophoresis is advantageous for being a non-invasive method for administering high concentrations of a medication, transdermally. The medication has to be charged and it is provided by repulsive electromotive force using a small electrical charge applied to an iontophoretic chamber containing a similarly charged active agent and its vehicle. To clarify, one or two chambers are filled with a solution containing an active ingredient and its solvent, termed the vehicle. The positively charged chamber, termed the anode will repel a positively charged chemical, while the negatively charged chamber, termed the cathode, will repel a negatively charged chemical into the skin. Unlike transdermal patches, this method relies on active transportation within an electric field, where, in the presence of an electric field, electromigration and electroosmosis are the dominant forces in the mass transport.

One may also use pulmonary administration in accordance with the present invention. In a particular embodiment, the route of administration is a route designed for optimum delivery of oxytocin, oxytocin analogs, oxytocin fragments or combinations thereof

The following examples serve to more fully describe the manner of using the above-described invention. It is understood that these examples in no way serve to limit the true scope of this invention, but rather are presented for illustrative purposes. All references cited herein are incorporated by reference in their entirety.

### EXAMPLES

### Example 1

### Dorsonasal Administration of Oxytocin

A pharmaceutical composition product comprising an aqueous solution formulation of oxytocin at a concentration sufficient to produce therapeutically effective plasma concentrations is used.

The methods being used for achieving dorsonasal delivery of oxytocin to individual patients are as follows:

(a) Oxytocin is administered to a first group of patients by an intranasal drip method.

(b) Oxytocin is administered to a second group using cotton swabs, the absorbent portions of which is saturated with oxytocin solution.

(c) Oxytocin is administered to patients in a third group by spraying the oxytocin solution into each nostril, using either a squeeze-type spray bottle or a metered-dose spray bottle.

The intranasal drip method may be based on the method described by Barre (1982, Headache 22:69-73), except that an oxytocin solution is used in place of the solution used by Bane. Approximately 0.75 milliliter to approximately 1.0 milliliter of the oxytocin solution is administered by way of each of the nostrils of each patient.

The cotton swab method comprises gently inserting cotton swabs, sequentially and bilaterally, into the nostrils of patients and urging the swabs dorsally until their absorbent portions contacted portions of nasal epithelium located dorsal to the middle conchae. Each swab is left in place for approximately one minute, and is then withdrawn. Approximately 0.5 milliliter of the oxytocin solution may be delivered to each nostril using this method.

Patients in a third group are administered oxytocin by spraying less than about 0.5 milliliter of the oxytocin solution into each of the patient's nostrils using either a sterile squeeze bottle or a sterile metered-dose spray bottle of known designs. The design and operation of each of these spray bottles are well known in the art.

Prior to administration of oxytocin, each patient is laid in a supine position with the patient's head hyperextended approximately 45 degrees and rotated approximately 30 degrees to the right side. In this position, an imaginary line extending from the region of the nasal epithelium overlying the SPG of the patient through the patient's left nostril is approximately vertical. Oxytocin is administered to the left nostril of the patient and then administered to each patient's right nostril after rotating the patient's head approximately 30 degrees to the left. In all groups, oxytocin may be administered to one of the nostrils of each patient.

Prior to oxytocin administration, and after treatment stuttering in each patient is rated, preferably, by a standard scale of the type used in the art. Follow-up of each patient's condition is attempted by direct contact or by telephone contact between six and eight hours post-treatment, between twenty-four and forty-eight hours post-treatment, and up to one week post-treatment.

Example 2

### Printed Patches of Oxytocin

Printed-patches are prepared by spotting droplets of oxytocin solution on a commercially appropriate backing liner in a predetermined pattern, hereinafter “printing”. The patches are allowed to dry at room temperature before use or storage.

Printing of oxytocin solution is performed using a microliter syringe, fitted with blunt needle. The printing pattern is digitally controlled by a dosing machine programmed to a predetermined pattern of 144 microliter sized droplets, in a 12×12 array. Total printing area is 1.44 cm2.

Oxytocin solutions at various concentrations, from 8.8 mg/ml to 17.6 mg/ml, are prepared for printing. Each solution is transferred to a 100 μl syringe. Air bubbles are removed from the syringe.

For preparing a patch with the desired quantity of oxytocin, the volume of each droplet is first calculated. Calculations of the syringe's plunger displacement required per one droplet are based on the concentration of oxytocin in the solution. The calculated range of displacement is fed into a program that controls the printing. Next, the backing layer film (DOW BLF2080™, The Dow Chemical Company, Mich., USA) is laid flat open (glossy side upwards) on a metal support. The syringe containing the oxytocin solution is loaded into the XYZ dosing machine, and oxytocin drops are dripped onto the liner. The patch is ready once the drops dry. Following completion of a 144 dots printed array, printing of a new array may be initiated. By the aforementioned technique up to 6 arrays on a 5.5×1.6″ backing liner may be prepared. Sections, 2×2cm², of the printed 144 dotted arrays are stored at 4° C. in close vials for future use.

### Example 3

### Evaluating Transdermal Permeation of Oxytocin

Transdermal permeation is measured in rats, males, 250 gr, Sprague Dawley, hypophysectomized (Harlan USA labs; n=24).

Fed rats, ad libitum, are divided into 4 groups as follows:

Group 1: Oxytocin transdermal patch was applied on perforated skin, treated with a voltage of 330V, frequency of 100 kHz, two bursts, 500-700 microsecond burst length, and no current limitation. The density of the microelectrode array is 100 microelectrodes/cm². Perforation is applied 2-5 times on each location, so the density of the micro channels is 200/cm².

Group 2: Oxytocin transdermal patch on intact skin.

Group 3: Subcutaneous (SC) injection of oxytocin.

Group 4: Untreated (control).

The transdermal groups (1 and 2) are treated prior to application of the patches (shaving, drying, baseline Trans Epidermal Water Loss measurement (TEWL; DERMALAB® Cortex Technology, Hadsund, Denmark). At the end of the study all animals are sacrificed by an IV overdose of sodium-pentobarbital (140 mg/kg).

Blood samples (0.5 ml/rat) are withdrawn at 6 time points, t=0, 2, 4, 6, 12 and 24 hours, from the tail end of anesthetized rats (20 mg/rat ketamine, intramuscular) and the level of oxytocin in the serum is evaluated.

The average concentrations of oxytocin in the serum are plotted in order to assess the pharmacokinetics of oxytocin in the blood, with respect to control (untreated rats). The results of the perforated skin are also compared to the results of the non-perforated/intact skin). For assessing bioavailability, AUC (the area under the curve which is expressed in units of ng*hr/ml) is calculated relatively to control, intact or SC values according to the following formula (exemplified for the Dose and SC groups):

(AUCgroup/Dosegroup)/(AUCSC/DoseSC)*100=Bioavailability (%).

While certain embodiments of the invention have been illustrated and described, it will be clear that the invention is not limited to the embodiments described herein.

Numerous modifications, changes, variations, substitutions and equivalents will be apparent to those skilled in the art without departing from the spirit and scope of the present invention as described by the claims, which follow.

1. A method of treating stuttering in a patient in need thereof, comprising administering to the patient a composition comprising a therapeutically effective amount of oxytocin, an oxytocin analog or a combination thereof.

2. The method of claim 1, wherein the oxytocin is an oxytocin analog selected from the group consisting of:
4-threonine-1-hydroxy-deaminooxytocin;
9-deamidooxytocin;
7-D-proline-oxytocin and its deamino analog;
(2,4-diisoleucine)-oxytocin;
deamino oxytocin analog;
1-deamino-1-monocarba-E12-[Tyr (OMe)]-OT(dCOMOT);
carbetocin;
Ile-conopressin;
atosiban;
[Thr4-Gly7]-oxytocin;
oxypressin;
deamino-6-carba-oxytoxin (dC60);
d[Lys(8)(5/6C-Flu)]VT;
d[Thr(4), Lys(8)(5/6C-Flu)]VT;
[HO(1)][Lys(8)(5/6C-Flu)]VT;
[HO(1)][Thr(4), Lys(8)(5/6C-Flu)]VT;
d[Om(8)(5/6C-Flu)]VT;
d[Thr(4), Om(8)(5/6C-Flu)]VT;
[HO(1)][Om(8)(5/6C-Flu)]VT;
[HO(1)][Thr(4), Om(8)(5/6C-Flu)]VT; and oxytocin fragments.

3. The method of claim 1, wherein the route of administration is selected from transmucosal administration, subcutaneous injection and transdermal administration.

4. The method of claim 3, wherein the route of administration is iontophoresis.

5. The method of claim 3, wherein the composition is administered by transmucosal administration selected from the group consisting of: buccal, nasal and sublingual.

6. The method of claim 5, wherein the composition is administered by nasal inhalation.

7. The method of claim 1, wherein the composition is administered in a modified release manner.

8. The method of claim 1, wherein said patient is treated by one or more therapies selected from speech therapy and behavioral therapy.

9. The method of claim 8, wherein the oxytocin is an oxytocin analog selected from the group consisting of:
4-threonine-1-hydroxy-deaminooxytocin;
9-deamidooxytocin;
7-D-proline-oxytocin and its deamino analog;
(2,4-diisoleucine)-oxytocin;
deamino oxytocin analog;
1-deamino-1-monocarba-E12-[Tyr (OMe)]-OT(dCOMOT);
carbetocin;
Ile-conopressin;
atosiban;
[Thr4-Gly7]-oxytocin;
oxypressin;
1 deamino-6-carba-oxytoxin (dC60);
d[Lys(8)(5/6C-Flu)]VT;
d[Thr(4), Lys(8)(5/6C-Flu)]VT;
[HO(1)][Lys(8)(5/6C-Flu)]VT;
[HO(1)][Thr(4), Lys(8)(5/6C-Flu)]VT;
d[Om(8)(5/6C-Flu)]VT;
d[Thr(4), Om(8)(5/6C-Flu)]VT;
[HO(1)][Om(8)(5/6C-Flu)]VT;
[HO(1)][Thr(4), Om(8)(5/6C-Flu)]VT and oxytocin fragments.

10. The method of claim 8, wherein the route of administration is selected from transmucosal administration, subcutaneous injection and transdermal administration.

11. The method of claim 10, wherein the route of administration is transdermal by means selected from the group consisting of: iontophoresis, hydrogels, patches, gels, and liposomes.

12. The method of claim 10, wherein the composition is administered by transmucosal administration selected from the group consisting of:
buccal, nasal and sublingual.

13. The method of claim 8, wherein the composition is administered by nasal or oral inhalation.

14. The method of claim 8, wherein the composition is administered in a modified release manner.

15. A method of treating speech impediments unrelated to psychiatric disorders or autism in a patient in need thereof, comprising administering to the patient a composition comprising a therapeutically effective amount of oxytocin or an analog thereof.

16-23. (canceled)

24. The method of claim 15, wherein the speech impediments unrelated to psychiatric disorders or autism are selected from: stuttering, dyspraxia, dysarthria, dysphasia and selective mutism.

25. The method of claim 15, wherein the route of administration is selected from transmucosal administration, subcutaneous injection and transdermal administration.

26. The method of claim 15, wherein said patient is treated by one or more therapies selected from speech therapy and behavioral therapy.
